(19) United States
(12) Patent Application Publication
(10) Pub. No.: US 2025/0360009 A1
(43) Pub. Date: Nov. 27, 2025

(54) DETACHABLE STENT ON EUS ACCESS DEVICE

(71) Applicant: Boston Scientific Scimed, Inc., Maple Grove, MN (US)

(72) Inventor: Alexander James Lammers, Brookline, MA (US)

(73) Assignee: Boston Scientific Scimed, Inc., Maple Grove, MN (US)

(21) Appl. No.: 19/237,628

(22) Filed: Jun. 13, 2025

Related U.S. Application Data
(60) Provisional application No. 63/650,488, filed on May 22, 2024.

(51) Int. Cl. A61F 2/95

(52) U.S. Cl. CPC A61F 2/9517; A61F 2002/9511; A61F 2220/0016; A61F 2250/0039; A61F 2250/0098

(57) ABSTRACT

A stent delivery system comprising an elongate tubular member having an outer surface and a lumen formed therein, a needle slidably disposed within the lumen of the elongate tubular member, a stent disposed on an outer surface of the needle distal of the elongate tubular member, and a suture loop attached to the distal end of the elongate member, wherein the suture loop is configured to be inserted through an opening in an outer surface of the stent to circumferentially surround the outer surface of the needle.

### CROSS-REFERENCE TO RELATED APPLICATIONS

This application claims the benefit of U.S. Provisional Application No. 63/659,552, filed Jun. 13, 2024, the disclosure of which is incorporated herein by reference in its entirety.

### TECHNICAL FIELD

The disclosure pertains to medical devices and methods for manufacturing medical devices. More particularly, the disclosure pertains to medical devices for delivering stents to the biliary tract and/or the pancreatic tract.

### BACKGROUND

A wide variety of intraluminal medical devices have been developed for medical use, for example use in the biliary tract. Some of these devices include guidewires, catheters, stents and the like. Of known medical devices, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example medical device includes a stent delivery system comprising an elongate tubular member having an outer surface and a lumen formed therein, a needle slidably disposed within the lumen of the elongate tubular member, a stent disposed on an outer surface of the needle, distal of the elongate tubular member, and a suture loop attached to the distal end of the elongate member, wherein the suture loop is configured to be inserted through an opening in an outer surface of the stent to circumferentially surround the outer surface of the needle.

Alternatively or additionally to the embodiment above, when the needle is retracted in a proximal direction past a proximal end of the stent, the suture loop is released and exits the opening in the outer surface of the stent.

Alternatively or additionally to any of the embodiments above, a distal end of the stent tapers to a diameter smaller than the diameter of a proximal end of the stent.

Alternatively or additionally to any of the embodiments above, the stent comprises one or more retention barbs configured to extend out from the outer surface of the stent at an angle relative to a longitudinal axis of the stent.

Alternatively or additionally to any of the embodiments above, the stent comprises a radiopaque marker disposed along an outer surface of the stent.

Alternatively or additionally to any of the embodiments above, the stent is formed of a flexible material.

Alternatively or additionally to any of the embodiments above, the elongate tubular member is formed of a stiffer material than the stent.

Alternatively or additionally to any of the embodiments above, the stent delivery system may further comprise a handle assembly, the handle assembly comprising an outer member, an inner member slidably disposed within the outer member, the inner member having a proximal end, a distal end, and a lumen extending therethrough, and a needle cap disposed on a proximal end of the needle, proximal of a proximal end of the outer member, wherein the distal end of the inner member is slidably attached to a proximal end of the elongate tubular member, such that the needle extends out from the lumen of the elongate tubular member to be slidably disposed within the lumen of the inner member.

Alternatively or additionally to any of the embodiments above, translation of the needle cap in a proximal direction relative to the outer member causes the needle to translate in a proximal direction.

Alternatively or additionally to any of the embodiments above, translation of the outer member relative to the inner member causes translation of the elongate tubular member, needle, and stent.

Alternatively or additionally to any of the embodiments above, the stent delivery system may further comprise a lure lock disposed on a proximal end of the needle cap.

Alternatively or additionally to any of the embodiments above, the outer member comprises a lock configured to lock the outer member in place relative to the inner member.

Alternatively or additionally to any of the embodiments above, the stent delivery system may further comprise a first handle portion disposed on the distal end of the inner member, and an outer catheter slidably disposed through a lumen of the first handle portion.

Alternatively or additionally to any of the embodiments above, the elongate tubular member, needle, and stent are slidably disposed within a lumen of the outer catheter.

Alternatively or additionally to any of the embodiments above, the first handle portion further comprises a lock, configured to fix the outer catheter in place relative to the inner member.

An example stent delivery system comprises a delivery assembly comprising an elongate tubular member having an outer surface and a lumen formed therein, a needle slidably disposed within the lumen of the elongate tubular member, a stent disposed on an outer surface of the needle, distal of the elongate tubular member, a suture loop attached to the distal end of the elongate member, wherein the suture loop is configured to be inserted through an opening in an outer surface of the stent to circumferentially surround the outer surface of the needle, and a handle portion comprising an outer member, an inner member slidably disposed within the outer member, the inner member having a proximal end, a distal end, and a lumen extending therethrough; and a needle cap disposed on a proximal end of the needle, proximal of a proximal end of the outer member, wherein the distal end of the inner member is slidably attached to a proximal end of the elongate tubular member, such that the needle extends out from the lumen of the elongate tubular member to be slidably disposed within the lumen of the inner member.

An example method of delivering a medical device comprises advancing a stent to a desired treatment location, wherein the stent comprises a lumen configured to receive a needle therethrough, the needle configured to extend out past a distal end of the stent to puncture a tissue, advancing the stent and needle to provide a puncture at the desired treatment location, withdrawing the needle proximal of the distal end of the stent, repositioning the stent within the desired treatment location, and withdrawing the needle proximal of the stent into a lumen of an elongate member, thereby deploying the stent into the desired treatment location.

Alternatively or additionally to any of the embodiments above, the proximal end of the stent is releasably attached to an elongate tubular member by a suture assembly.

Alternatively or additionally to any of the embodiments above, the suture assembly comprises a suture loop attached to a distal end of the elongate member, wherein the suture loop is configured to be inserted through an opening in an outer surface of the stent to circumferentially surround an outer surface of the needle.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a side view of an example medical device;

FIG. 2A illustrates a perspective view of the device of FIG. 1 in use;

FIG. 2B illustrates a perspective view of the device of FIG. 1 in use;

FIG. 2C illustrates a perspective view of the device of FIG. 1 in use;

FIG. 3A illustrates an exemplary stent detachment mechanism;

FIG. 3B illustrates an exemplary stent detachment mechanism.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

It will be understood that the dimensions described in association with the above figure are illustrative only, and that other dimensions of slits and filter sheaths are contemplated. The materials that can be used for the various components of the stent delivery system for capturing lesion particles (and/or other systems or components disclosed herein) and the various elements thereof disclosed herein may include those commonly associated with medical devices. For simplicity purposes, the following discussion refers to the stent delivery system (and variations, systems or components disclosed herein). However, this is not intended to limit the devices and methods described herein, as the discussion may be applied to other elements, members, components, or devices disclosed herein.

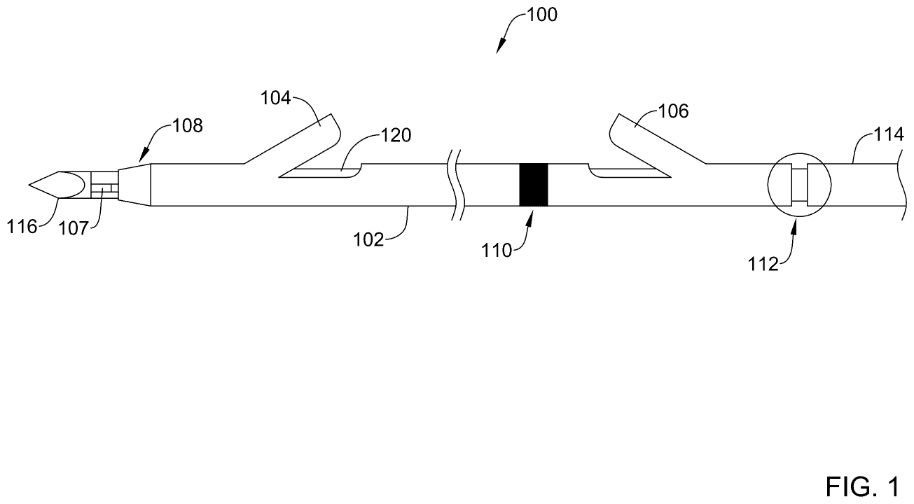

FIG. 1 is a side view of an illustrative biliary access device. While the disclosure concentrates on gaining access to the biliary and pancreatic ducts and ultimately delivering a medical device, it will be appreciated that this is merely illustrative as the concepts described herein are equally applicable to gaining access and delivering a medical device to any of a variety of different regions or systems of the human anatomy.

The illustrative biliary access device 100 may be used to access the biliary and pancreatic ducts as well as related anatomy. The biliary device facilitates tissue puncture and device placement, such as stent 102. The biliary access device 100 provides the ability to puncture through a tissue into a target site, partially withdraw the needle or sharp 120, and reposition a medical device or stent 102 without risk of puncturing or further damaging the surrounding tissue with the needle's sharp end 116. The continued removal of the needle or sharp 120 then releases the stent 102 or medical device into the desired treatment location. The device 100 may be used under fluoroscopy, ultrasound guidance, or even direct visualization.

The device 100 includes a proximal access cannula 114 and detachable stent 102. The stent 102 and access cannula 114 are coaxial with one another and are adjacent such that a distal end of the proximal access cannula 114 abuts a proximal end of the stent 102 to form a detachment location 112, or a separation point between the two elongate members 102, 114. The stent may include anti-migration barbs 104 and 106 which extend out from the outer surface of the stent. Anti-migration barbs 104 and/or 106 may be configured to prevent the stent from being displaced once disposed in the desired treatment location. The anti-migration barbs 104 and 106 may be of any shape or style known in the art e.g. shouldered ends, barbs, knurls, wedges or the like. The stent 102 may also include one or more radiopaque markers 110 along its length configured to allow a user to visualize the stent position throughout the procedure. The stent 102 may also include a tapered distal end 108, allowing for ease of movement of the stent 102 within a treatment location after the initial puncture has been completed. The tapered end 108 may also reduce the amount of puncture force required to pass the needle and stent system through a tissue wall after initial puncture from the needle.

The needle or sharp 120 extends through a lumen of the proximal access cannula 114, through a lumen of the stent 102, and is slidable through both lumens. In some embodiments, the needle 120 may be hollow to allow for injection or aspiration of materials to and from a target location. The needle or sharp 120 may include an injectable window 107 for injection of fluids into a duct e.g. contrast for visualization. The injection window 107. This window 107 may also allow for aspiration of bile samples or other fluids from a duct. The window 107 may be fully exposed, or partially covered by the stent, and may be disposed at any longitudinal position along the length of the needle or sharp 120. The distal tapered end 108 may be tapered to a diameter greater than or equal to the diameter of the needle 120, or the lumen through which the needle passes. The sharp distal end 116 of the needle is configured to extend out from the tapered distal end 108 of the stent 102.

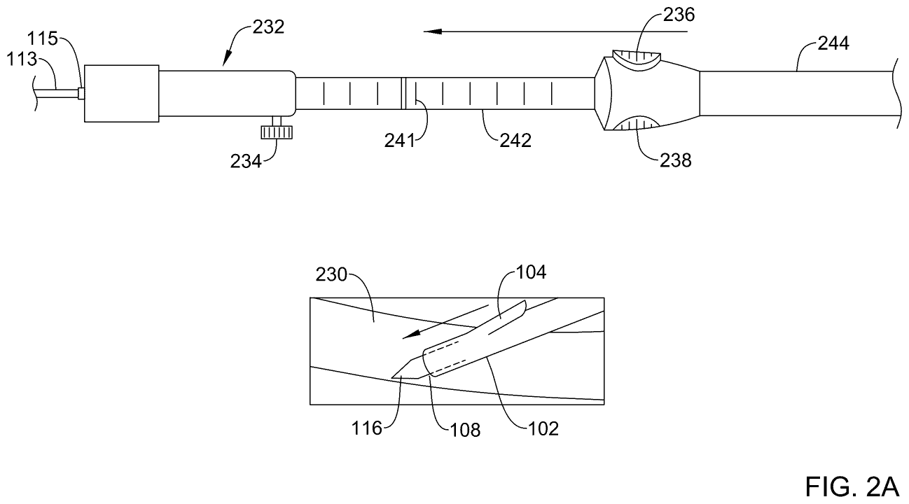

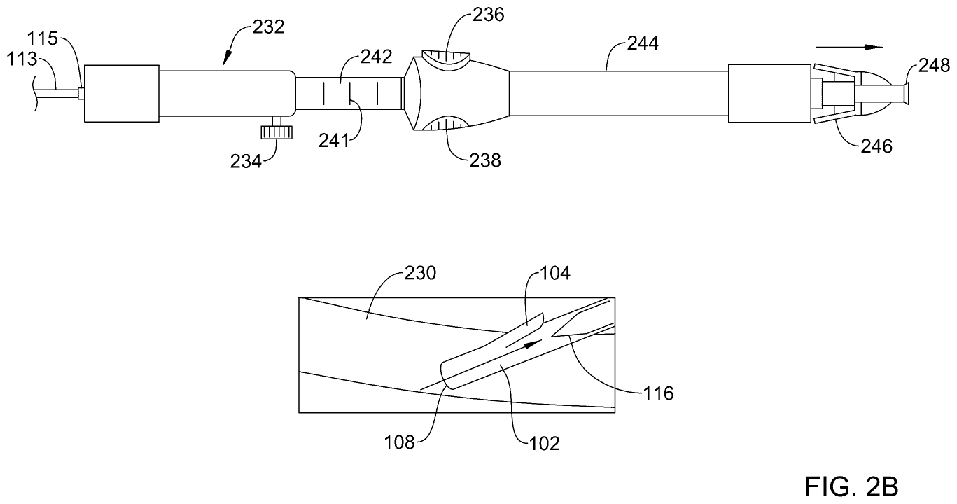

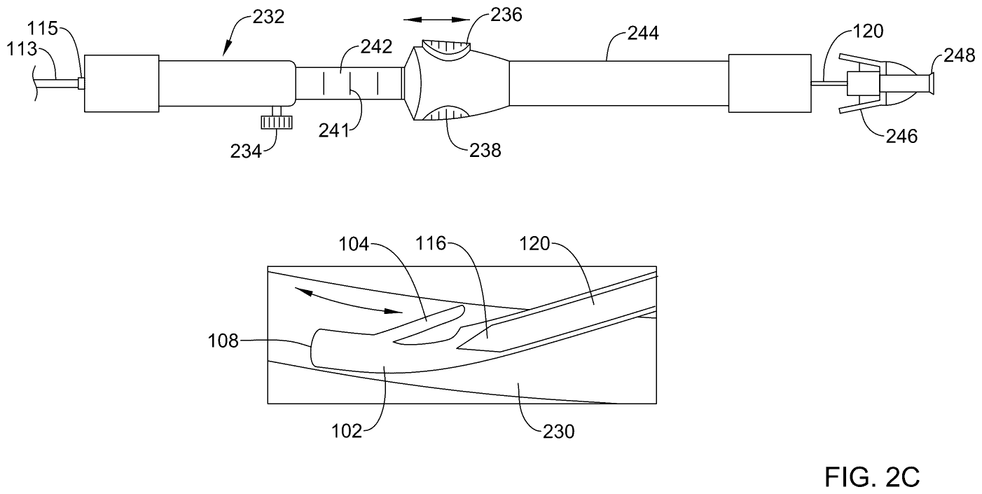

As shown in FIGS. 2A-2C, the biliary access device 100 includes a handle assembly. The handle assembly includes first handle portion 232 attached to an outer catheter 113. In some embodiments, the first handle portion 232 is slidably attached to outer catheter 113 while outer catheter 113 is fixedly attached to inner member 242. Thus, when a user adjusts the position of the first handle portion 232 relative to the inner member 242 it shortens (or lengthens as may be the case) the amount of the outer catheter 113 extending out from the fisrt handle portion 232.

The first handle portion 232 may include a lure lock 115 or other attachment device configured to attach the outer cannula 113 and the first handle portion 232 to an endoscope (e.g., a lure on an echoendoscope, or the like). The handle further includes an inner member 242 slidably attached to a proximal end of the first handle portion 232 and slidably disposed within an outer member 244. The outer catheter 113 extends through a lumen of the first handle portion 232 and lumen of the inner member 242, which as stated can in some embodiments be fixedly attached to the outer catheter 113. The proximal access cannula 114 extends proximally through a lumen of the outer catheter 113, a lumen of the first handle portion 232 and a lumen of the inner member 242. A proximal end of the proximal access cannula 114 is fixedly attached to the outer member 244. Said differently, the outer catheter 113 surrounds the proximal access cannula 114. The proximal access cannula 114 and stent 102 are slidable within a lumen of the outer catheter 113.

The outer member 244 is slidable relative to the inner member 242, to effectively actuate the proximal access cannula 114 relative to the outer cannula 113. The inner member 242 may include a scale or markings 241 along a length thereof to be used as a guide while adjusting the length of the handle assembly. For example, the scale can be configured to provide an indication of the distance of puncture by the needle into tissue. In some embodiments, the markings 241 may be notches that engage with a locking mechanism. The outer member 244 includes a button 236 which unlocks the outer member 244 relative to the inner member 242, and allows for length adjustment of the handle. In some embodiments, the button may include a spring mechanism configured to insert teeth into the notches or markings 241 along the length of the inner member 242, effectively preventing movement of the outer member 244 when the button is not compressed. As shown in FIG. 2A, translation of the outer member 244 in the distal direction corresponds to translation of the stent 102, proximal access cannula 114, and needle 116 in the proximal direction. Said differently, as a user pushes the outer member 244 distally, all parts of the biliary access device 100 translate distally.

The first handle portion 232 may further include a lock 234 configured to lock the first handle portion 232 in place relative to the inner member 242. For example, a physician may advance or retract the first handle portion 232 to affect the working length of the outer catheter 113, and then lock the first handle portion and outer catheter 113 in place. A distal end of the outer catheter 113 may be advanced up against a tissue wall (e.g. bile duct, gastric wall or the like) and visualized prior to puncturing the tissue. The distal end of the outer catheter 113 may be blunt to prevent damage or puncture to tissue during positioning of the device. Once the outer catheter is in place, a physician may advance the needle or sharp 120 past the distal end of the outer catheter 113 to create a puncture.

With continued reference to FIG. 2A, as the outer member 244 is translated forward, the proximal access cannula 114, stent 102, and distal end of the needle 116 translate forward to puncture through a tissue wall and enter a target location 230. Though not shown, the proximal access cannula 114 and stent 102 are still connected at the detachment location 112 (FIG. 1). As such, the proximal access cannula 114 and stent 102 move as one piece. Said differently, as the stent 102 of FIG. 2A moves forward, the proximal access cannula also moves forward in response to actuation of the outer member 244. There may be a grip or textured portion 238 provided on the outer member 244 to assist a user in actuating the outer member 244.

With reference to FIG. 2B, the handle assembly further includes a needle cap 246. The needle cap is attached to the proximal end of the needle 120. The needle cap may be releasably connected to the proximal end of the outer member 244 such that it remains in place relative to the outer member 244 throughout actuation of the handle assembly, but can be removed from the handle assembly for independent actuation of the needle cap relative to the outer member 244. In some embodiments, the needle cap 246 may include an injection port in fluid communication with the needle. The needle cap 246 may also include a lure attachment 238 to allow for attaching accessory medical devices to the needle cap 246 e.g. syringe, suction, or the like. The needle cap 246 is detachable from the handle assembly, and may be withdrawn from the handle assembly in the proximal direction. As shown in FIG. 2B, Translation of the needle cap 246 corresponds to translation of the needle 120 within the stent 102 and proximal access cannula 114. The stent 102 and proximal access cannula 114 do not move in response to movement of the needle cap 246. As discussed in more detail above, the sharp end of the needle 116, can be withdrawn past the distal end of the stent 108 such that the sharp is within the stent and is unable to puncture or damage surrounding tissue.

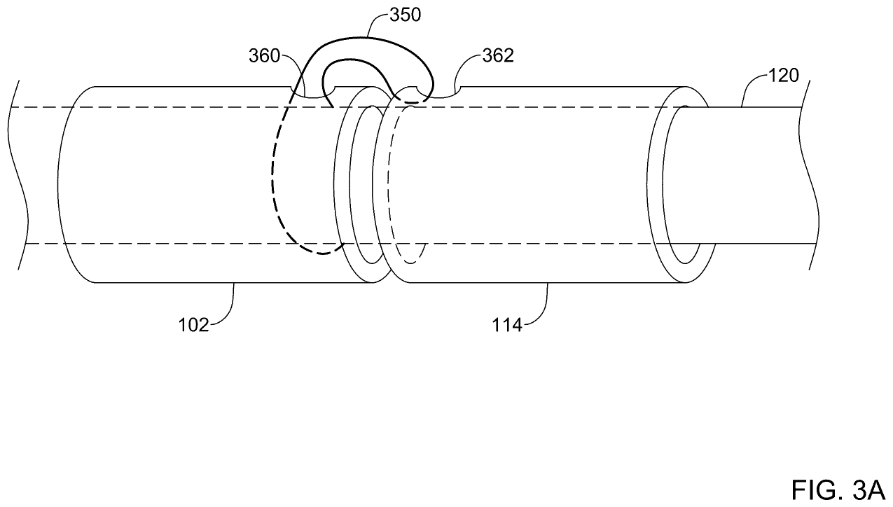

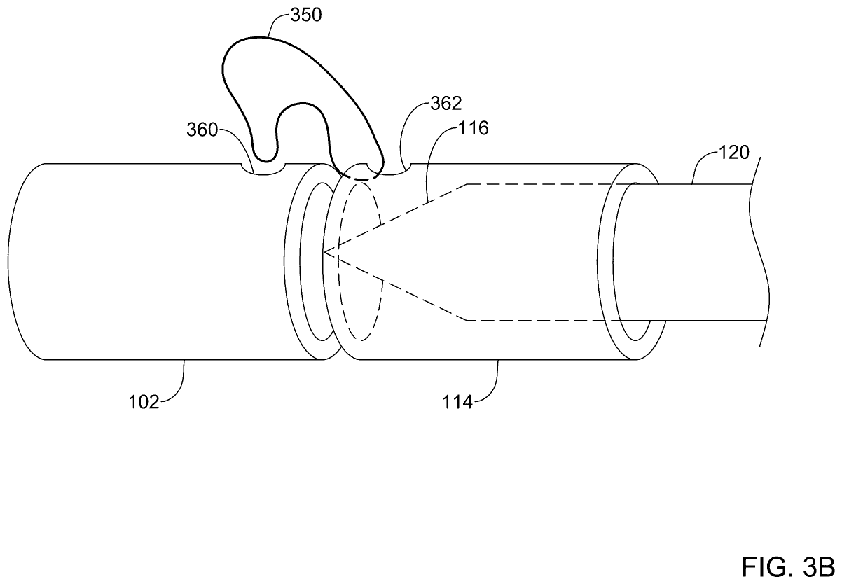

As shown in FIG. 2C, the stent 102 may be repositioned within the target location 230 after the needle 120 has been partially retracted. The outer member 244 can be actuated in the proximal and distal directions to advance or retract the stent 102. As discussed in more detail above, the distal end of the stent 108 can be tapered (FIG. 1) to reduce the risk of puncturing or damaging the tissue around the stent 102 during positioning. The tapered end 108 may also facilitate ease of repositioning the stent 102, and allow for the stent 102 to slide smoothly through a duct 230 without damaging or catching on surrounding tissue. After the stent 102 is in a desired position, the needle 120 may be completely withdrawn from the delivery system by withdrawing the needle cap 246 proximally. Removal of the needle 120 releases the stent 102 from the proximal access cannula 114 (FIG. 3B) thereby leaving the stent 102 in place within the treatment location 230.

As shown in FIG. 3A, the stent 102 and proximal access cannula 114 are connected at a detachment location 112 (FIG. 1) by a suture 350. The suture 350 is attached to a distal end of the proximal access cannula 114 through an opening in the outer surface 362. The suture 350 may be tied, looped, or otherwise connected to the proximal access cannula 114 permanently or releasably. The other end of the suture 350 is configured to be inserted through an opening in an outer surface of the stent 360. Within the lumen of the stent 102, the suture 350 circumferentially surrounds an outer surface of the needle 120. Said differently, when the needle 108 extends through lumens of both the stent 102 and proximal access cannula 114, it is also fed through the suture loop 350. The suture 350 is shaped and sized such that it fits around the needle and secures the stent 102 to the proximal access cannula 114.

With reference to FIG. 3B, the suture 350 is releasably attached to the needle 120. When the needle 120 is withdrawn proximal of the stent 102, the suture 350 is released from around the needle 120. Said differently, the needle 108 slides out of the suture loop 350, leaving the suture in place within the stent 102. When the suture 350 is no longer secured around the needle 120, the suture 350 is free to exit the opening in the outer surface of the stent 102, such that the stent 102 is no longer connected to the proximal access cannula 114. The biliary access device 100 can then be withdrawn from the treatment location 230, leaving the stent 102 in place within the target location 230 e.g. biliary duct or the like.

In some embodiments, the stent delivery system (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the stent delivery system (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the stent delivery system (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stent delivery system (and variations, systems or components thereof disclosed herein) to achieve the same result.

In some embodiments, the stent delivery system (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the stent delivery system (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent delivery system, comprising:
an elongate tubular member having an outer surface and a lumen formed therein;
a needle slidably disposed within the lumen of the elongate tubular member;
a stent disposed on an outer surface of the needle, distal of the elongate tubular member; and
a suture loop attached to the distal end of the elongate member, wherein the suture loop is configured to be inserted through an opening in an outer surface of the stent to circumferentially surround the outer surface of the needle.

2. The stent delivery system of clam 1, wherein when the needle is retracted in a proximal direction past a proximal end of the stent, the suture loop is released and exits the opening in the outer surface of the stent.

3. The stent delivery system of claim 1, wherein a distal end of the stent tapers to a diameter smaller than the diameter of a proximal end of the stent.

4. The delivery system of claim 1, wherein the stent comprises one or more retention barbs configured to extend out from the outer surface of the stent at an angle relative to a longitudinal axis of the stent.

5. The delivery system of claim 1, wherein the stent comprises a radiopaque marker disposed along an outer surface of the stent.

6. The stent delivery system of claim 1, wherein the stent is formed of a flexible material.

7. The stent delivery system of claim 6, wherein the elongate tubular member is formed of a stiffer material than the stent.

8. The stent delivery system of claim 1, further comprising a handle assembly, the handle assembly comprising:
an outer member;
an inner member slidably disposed within the outer member, the inner member having a proximal end, a distal end, and a lumen extending therethrough; and
a needle cap disposed on a proximal end of the needle, proximal of a proximal end of the outer member,
wherein the distal end of the inner member is slidably attached to a proximal end of the elongate tubular member, such that the needle extends out from the lumen of the elongate tubular member to be slidably disposed within the lumen of the inner member.

9. The stent delivery system of claim 8, wherein translation of the needle cap in a proximal direction relative to the outer member causes the needle to translate in a proximal direction.

10. The stent delivery system of claim 9, wherein translation of the outer member relative to the inner member causes translation of the elongate tubular member, needle, and stent.

11. A stent delivery system comprising:
a delivery assembly comprising:
an elongate tubular member having an outer surface and a lumen formed therein;
a needle slidably disposed within the lumen of the elongate tubular member;
a stent disposed on an outer surface of the needle, distal of the elongate tubular member;
a suture loop attached to the distal end of the elongate member, wherein the suture loop is configured to be inserted through an opening in an outer surface of the stent to circumferentially surround the outer surface of the needle; and
a handle portion comprising:
an outer member;
an inner member slidably disposed within the outer member, the inner member having a proximal end, a distal end, and a lumen extending therethrough; and
a needle cap disposed on a proximal end of the needle, proximal of a proximal end of the outer member,
wherein the distal end of the inner member is slidably attached to a proximal end of the elongate tubular member, such that the needle extends out from the lumen of the elongate tubular member to be slidably disposed within the lumen of the inner member.

12. The stent delivery system of claim 11, wherein translation of the needle cap in a proximal direction relative to the outer member causes the needle to translate in a proximal direction.

13. The stent delivery system of claim 12, wherein translation of the outer member relative to the inner member causes simultaneous translation of the elongate tubular member, needle, and stent.

14. The stent delivery system of clam 11, wherein when the needle is retracted in a proximal direction past a proximal end of the stent, the suture loop is released and exits the opening of the outer surface of the stent.

15. The stent delivery system of claim 11, wherein a distal end of the stent tapers to a diameter smaller than the diameter of a proximal end of the stent.

16. The stent delivery system of claim 11, wherein the elongate tubular member is formed of a stiff material.

17. The stent delivery system of claim 11, wherein the stent is formed of a flexible material.

18. A method of delivering a medical device, the method comprising:
advancing a stent to a desired treatment location, wherein the stent comprises a lumen configured to receive a needle therethrough, the needle configured to extend out past a distal end of the stent to puncture a tissue;
advancing the stent and needle to provide a puncture at the desired treatment location;
withdrawing the needle proximal of the distal end of the stent;
repositioning the stent within the desired treatment location; and
withdrawing the needle proximal of the stent into a lumen of an elongate member, thereby deploying the stent into the desired treatment location.

19. The method of claim 18, wherein the proximal end of the stent is releasably attached to an elongate tubular member by a suture assembly.

20. The method of claim 19, wherein the suture assembly comprises a suture loop attached to a distal end of the elongate member, wherein the suture loop is configured to be inserted through an opening in an outer surface of the stent to circumferentially surround an outer surface of the needle.
